(19) United States
(12) Patent Application Publication
(10) Pub. No.: US 2015/0342661 A1
(43) Pub. Date: Dec. 3, 2015

(54) Integrated Treatment System

(71) Applicant: VENUS CONCEPT LTD, Karmiel (IL)

(72) Inventor: Orit RON EDOUTE, Tel Aviv (IL)

(21) Appl. No.: 14/650,680

(22) PCT Filed: Oct. 2, 2014
(86) PCT No.: PCT/IL14/50867

§ 371 Date: Jun. 9, 2015

Related U.S. Application Data
(60) Provisional application No. 61/888,535, filed on Oct. 9, 2013.

(51) Int. Cl. A61B 18/04; A61M 37/00

(52) U.S. Cl. CPC A61B 18/04; A61M 37/0015; A61M 2037/0061; A61M 2037/0023

(57) ABSTRACT

The present invention provides a device for improving the cosmetic appearance of the skin, comprising a housing, said housing comprising: a. N electrodes, where N is greater than two, said electrodes configured to transmit RF energy to said skin so as to provide RF treatment to said skin; b. energy generating means for generating N/2 independent signals of predetermined waveforms, frequencies, and amplitudes; each of said N electrodes in independent communication with said energy generating means; c. at least one capsule, adapted to contain at least one medicament; said capsule within said housing; and d. M needles in fluid communication with said capsule; where M is greater than one; wherein said medicament is applied to said skin through at least one of said M needles either prior to or during said treatment, such that said medicament is applied in conjunction with said treatment.

### FIELD OF THE INVENTION

The present invention generally pertains to a system and method for treating skin conditions by applying medicament and RF treatment in conjunction with each other.

### BACKGROUND OF THE INVENTION

Improving the appearance of the skin has been the goal of many esthetic products and procedures for many years, since a tight skin, without wrinkles or cellulite, has a younger and more appealing appearance. Apart from age related changes, the skin also suffers from exposure to chemical and physical injuries, such as tobacco, cosmetics, esthetics and radiation from the sun and other sources. Those factors contribute to the decrease in collagen production, to reduced elasticity, and the appearance of wrinkles.

The skin and muscles of the face are structured differently than other places on the body. One side of the facial muscles is connected to the bone and the other to the skin. As the muscle deteriorates through the aging process, the attached facial skin loses it elasticity. Loss of elasticity causes the skin to sag and wrinkle. Strengthening relevant muscle groups restores and maintains the original shape and contour of the muscles. As facial muscles get stronger, they get shorter and flatter, causing the attached skin to become firmer, and smoothing wrinkles, improving facial appearance. Additionally, a contracting muscle's blood supply is 10 times greater than a muscle at rest. This fresh blood supply delivers vital oxygen and nutrients to the skin, revitalizing the tissue.

The most common method of heating the dermis, is the use of RF radiation, applied by antenna or electrodes. For example, WO98005380 discloses a method of tightening skin using an RF electromagnetic energy delivery device. However, the manner (and specifically, the protocol) in which the RF is transmitted to the region of interest is highly important. Some methods will have no effect and others may have the opposite effect.

U.S. Pat. Appl. 20100016850 discloses an invention that demonstrates that application of electrical currents of about 1 milliampere, and voltages greater than 4V have a much greater esthetic effect on the skin than application of lower currents and voltages known in the prior art. This application further discloses that simultaneous application of electrical currents on the skin yields better esthetic results.

It can be desirable to include medicaments during the treatment. Medicaments can include, but are not limited to, a local anesthetic to reduce the discomfort of the RF treatment, medicaments intended to treat the skin condition, a dye to absorb light in embodiments that use optical means for effecting deep tissue diathermy, a muscle relaxant, etc.

RF energy can improve the efficacy of transdermal delivery of medication.

Thus, means for providing RF (or in general, any kind of energy) to the region of treatment (e.g., the skin) in conjunction with transdermal delivery of medication remains a long-felt, yet unmet, need.

### SUMMARY OF THE INVENTION

It is one object of the present invention to disclose a device for improving the cosmetic appearance of the skin, comprising a housing, said housing comprising:
a. N electrodes, where N is greater than two, said electrodes configured to transmit RF energy to said skin so as to provide RF treatment to said skin;
b. energy generating means for generating N/2 independent signals of predetermined waveforms, frequencies, and amplitudes; each of said N electrodes in independent communication with said energy generating means;
c. at least one capsule, adapted to contain at least one medicament; said capsule within said housing; and
d. M needles in fluid communication with said capsule; where M is greater than one;
   wherein said medicament is applied to said skin through at least one of said M needles either prior to or during said treatment, such that said medicament is applied in conjunction with said treatment.

It is another object of the present invention to disclose the device, wherein said energy is selected from a group consisting of RF, Intense pulsed light (IPL), laser and any combination thereof.

It is another object of the present invention to disclose the device, additionally comprising control means for controlling the output of said RF generating means, said control means in communication with said RF generating means,

It is another object of the present invention to disclose the device, wherein said delivery of said medicament in conjunction with said RF is selected from a group consisting of: medicament is delivered, then RF is applied, RF and medicament are applied simultaneously, RF is applied and then medicament, and any combination thereof.

It is another object of the present invention to disclose the device, wherein said electrodes provide fractional RF treatment of said skin, said RF energy being applied to said skin through a subset of said N electrodes, said subset of said N electrodes changing with time.

It is another object of the present invention to disclose the device, wherein said needles provide fractional medicament treatment of said skin, the medicament being applied to the skin through a subset of said M needles, said subset of said M needles changing with time.

It is another object of the present invention to disclose the device, wherein said subset of said N electrodes and said subset of said M needles change in conjunction with each other.

It is another object of the present invention to disclose the device, wherein the distal end of said electrode has a shape selected from a group consisting of: pointed, cylindrical, rounded, conic, and truncated conic.

It is another object of the present invention to disclose the device, wherein said capsule is frangible.

It is another object of the present invention to disclose the device, wherein said medicament is squeezed from said housing onto said skin.

It is another object of the present invention to disclose the device, wherein said medicament flows from said housing onto said skin.

It is another object of the present invention to disclose the device, wherein at least a portion of said capsule is adapted such that at least a portion thereof ruptures upon application of at least one of: at least one predetermined pressure on at least a portion of the exterior of said housing, rotation of one part of said housing with respect to another part of said housing and any combination thereof.

It is another object of the present invention to disclose the device, wherein said capsule is adapted such said rupture induces fluid connection between the interior of said capsule and said skin via at least one said needle.

It is another object of the present invention to disclose the device, wherein at least one of the following is true:
a. at least one of said needles penetrates at least one layer of said skin;
b. none of said needles penetrates any layer of said skin;
c. at least one of said electrodes ablates a portion of at least one layer of said skin.

It is another object of the present invention to disclose the device, wherein any of the following is held true:
a. each of said predetermined frequencies is between about 1 Hz and about 100 MHz;
b. RF signals are transmitted in either a continuous mode or in pulses; when the RF is applied in pulses, the length of said pulses is between about 0.01 μs and about 1 ms; length of said pulses is between about 0.1 and about 1000 ms;
c. additionally comprises means adapted to apply Pulsed Electromagnetic Field Therapy (PEMF);
d. additionally comprises temperature measuring means adapted to measure the temperature of the surface of said skin; said temperature measuring means comprises at least one sensor chosen from a group consisting of impedance meter adapted to measure impedance across at least one of pair of said RF electrodes; thermal sensor; thermometer; and any combination thereof; said temperature measuring means either come into contact with the skin or are not in contact with the skin; said control means are programmed to regulate the amount of RF energy transmitted to said skin such that the temperature of said skin remains within a predetermined range; said predetermined range is between ambient temperature and 42° C.; said predetermined range is between 30° C. and 100° C.;
e. said electrodes are disposed about the distal end of said housing in a geometry chosen from a group consisting of linear; zigzag; on the perimeter of a shape chosen from substantially polygonal, circular, oval, or irregular; within the area of a shape chosen from substantially polygonal, circular, oval, or irregular; and any combination of the above;
f. the power transmitted by said RF electrodes and said RF generating means to said skin is between 1 W and 700 W;
g. said cosmetic improvement is chosen from a group consisting of skin rejuvenation, reduction of the number of wrinkles, reduction of the depth of wrinkles, reduction of cellulite, skin tightening, circumferential reduction, and any combination of the above;
h. additionally comprising cooling means adapted to cool said skin;
i. said cooling means are chosen from a group consisting of a Peltier effect cooling device, irrigation with cool water, and means for blowing air across the skin;
j. said RF electrodes are further adapted to provide heat to said skin;
k. additionally comprising means for massaging said skin; and,
l. at least one of said RF electrodes comprises a hypodermic syringe for penetrating into subcutaneous tissue.

It is another object of the present invention to disclose the device, additionally comprising a deep tissue diathermy device.

It is another object of the present invention to disclose the device, wherein any of the following is held true:
a. said deep tissue diathermy device is chosen from a group consisting of any devices emitting RF radiation and any other means adapted for producing electrical current absorbed by subcutaneous tissue.
b. said deep tissue diathermy device further comprises:
   i. at least one electrical output device adapted to generate RF electromagnetic energy; and,
   ii. at least two electrodes electrically coupled to said electrical output device and placed on said skin region, wherein all said electrodes are adapted to simultaneously apply said RF energy to said skin.
c. said deep tissue diathermy device further comprises:
   i. at least one electrical output device adapted to generate electrical current; and,
   ii. at least two electrodes electrically coupled to said electrical output device and placed on said skin region, wherein all of said electrodes are adapted to simultaneously apply said electrical current to said skin.
d. said deep tissue diathermy device is chosen from a group consisting of acoustic (e.g., ultrasonic) diathermy devices, optical diathermy devices, electromagnetic induction diathermy devices, devices for producing sound waves, ultrasonic diathermy devices, and devices for direct application of heat.
e. said deep tissue diathermy device is an optical device adapted to emit light in wavelengths absorbed by subcutaneous tissue such that said subcutaneous tissue is heated;
f. said deep tissue diathermy device is a device for producing sound waves adapted to emit sound waves of a frequency absorbed by the subcutaneous tissue such that said subcutaneous tissue is heated;
g. said control means are adapted to monitor physical tissue parameters and to change at least one of (a) the amount of heat applied and (b) the form of said RF in response to the values of said physical tissue parameters;
h. said control means further comprise:
   i. processing means adapted to store in a communicable database predetermined parameters defining safe and unsafe treatment parameters, said parameters chosen from a group consisting of time of said treatment, the temperature of said skin, frequency, power, energy applied by said RF generating means, depth to which said device operates, magnetic field intensity, tissue impedance, specific absorption rate, superficial muscle contractions and any combination thereof;
   ii. sensing means adapted to sense electromagnetic radiation and heat radiation parameters chosen from a group consisting of time of said treatment, temperature of said tissue, frequency, power, intensity of ultrasound irradiation, energy applied by said RF generating means, depth to which said device operates, magnetic field intensity, tissue impedance, specific absorption rate, superficial muscle contractions and any combination thereof; and,
   iii. regulating means adapted to stop the operation of said device if said parameters are determined to be unsafe.

It is another object of the present invention to disclose the device, wherein said control means additionally comprise a feedback mechanism, adapted to change said RF signal according to predetermined medical needs, and comprising:
a. sensing means adapted to monitor electrotherapy parameters related to the level of skin rejuvenation and viability;
b. processing means, adapted to determine the degree of esthetic improvement in at least one tissue parameter related to the level of skin rejuvenation and vitality; and,
c. regulating means adapted to stop the operation of said device when said degree of esthetic improvement reaches a predetermined value.

It is another object of the present invention to disclose the device, wherein any of the following is held true:
a. said electrotherapy parameters are chosen from a group consisting of dermal tensile forces, tissue impedance, muscle contraction forces, skin elasticity, and any combination thereof.
b. said at least one tissue parameter is chosen from a group consisting of dermal tensile forces, tissue impedance, muscle contraction forces, skin elasticity, and any combination thereof.
c. wherein said sensing means are adapted to sense electrotherapy parameters are chosen from a group consisting of time of said treatment, temperature of said tissue, frequency, power, tissue impedance, superficial muscle contractions and any combination thereof.
d. said processing means are adapted to store in a communicable database predetermined parameters defining safe and unsafe treatment conditions; said predetermined parameters are chosen from a group consisting of time of said treatment, temperature of said tissue, frequency, power, tissue impedance, superficial muscle contractions, and any combination thereof.

It is another object of the present invention to disclose a device for improving the cosmetic appearance of the skin, comprising a housing, said housing comprising:
a. N electrodes, where N is greater than two, said electrodes configured to transmit RF energy to said skin so as to provide treatment to said skin;
b. energy generating means for generating N/2 independent signals of predetermined waveforms, frequencies, and amplitudes; each of said N electrode in independent communication with said generating means; and
c. at least one capsule, adapted to contain at least one medicament; said capsule within said housing; at least one of said RF electrodes is in fluid communication with said capsule;
   wherein at least one of said N electrodes comprises a distal penetrating means adapted to penetrate said skin so as to deliver said medicament;
   further wherein said medicament is applied to said skin through at least one of said distal penetrating means either prior to or during said treatment, such that said medicament is applied in conjunction with said treatment.

It is another object of the present invention to disclose the device, wherein said energy is selected from a group consisting of RF, Intense pulsed light (IPL), laser and any combination thereof.

It is another object of the present invention to disclose the device, additionally comprising control means for controlling the output of said RF generating means, said control means in communication with said RF generating means,

It is another object of the present invention to disclose the device, wherein said delivery of said medicament in conjunction with said RF is selected from a group consisting of: medicament is delivered, then RF is applied, RF and medicament are applied simultaneously, RF is applied and then medicament, and any combination thereof.

It is another object of the present invention to disclose the device, wherein said electrodes provide fractional RF treatment of said skin, said RF energy being applied to the skin through a subset of said N electrodes, said subset of said N electrodes changing with time.

It is another object of the present invention to disclose the device, wherein said penetrating means provide fractional medicament treatment of said skin, the medicament being applied to the skin through a subset of said penetrating means, said subset of said penetrating means changing with time.

It is another object of the present invention to disclose the device, wherein said subset of said N electrodes and said subset of said penetrating means change in conjunction with each other.

It is another object of the present invention to disclose the device, wherein the distal end of said electrode has a shape selected from a group consisting of: pointed, cylindrical, rounded, conic, and truncated conic.

It is another object of the present invention to disclose the device, wherein said capsule is frangible.

It is another object of the present invention to disclose the device, wherein said medicament is squeezed from said housing onto said skin.

It is another object of the present invention to disclose the device, wherein said medicament flows from said housing onto said skin.

It is another object of the present invention to disclose the device, wherein at least a portion of said capsule is adapted such that at least a portion thereof ruptures upon application of at least one of: at least one predetermined pressure on at least a portion of the exterior of said housing, rotation of one part of said housing with respect to another part of said housing and any combination thereof.

It is another object of the present invention to disclose the device, wherein said capsule is adapted such that said rupture induces fluid connection between the interior of said capsule and said skin via said at least one penetrating means.

It is another object of the present invention to disclose the device, wherein at least one of the following is true:
a. at least one of said penetrating means penetrates at least one layer of said skin;
b. none of said penetrating means penetrates any layer of said skin;
c. at least one of said electrodes ablates a portion of at least one layer of said skin.

It is another object of the present invention to disclose the device, wherein any of the following is held true:
a. each of said predetermined frequencies is between about 1 Hz and about 100 MHz;
b. RF signals are transmitted in either a continuous mode or in pulses; when the RF is applied in pulses, the length of said pulses is between about 0.01 μs and about 1 ms; length of said pulses is between about 0.1 and about 1000 ms;
c. additionally comprises means adapted to apply Pulsed Electromagnetic Field Therapy (PEMF);
d. additionally comprises temperature measuring means adapted to measure the temperature of the surface of said skin; said temperature measuring means comprises at least one sensor chosen from a group consisting of impedance meter adapted to measure impedance across at least one of pair of said RF electrodes; thermal sensor; thermometer; and any combination thereof; said temperature measuring means either come into contact with the skin or are not in contact with the skin; said control means are programmed to regulate the amount of RF energy transmitted to said skin such that the temperature of said skin remains within a predetermined range; said predetermined range is between ambient temperature and 42° C.; said predetermined range is between 30° C. and 100° C.;
e. said electrodes are disposed about the distal end of said housing in a geometry chosen from a group consisting of linear; zigzag; on the perimeter of a shape chosen from substantially polygonal, circular, oval, or irregular; within the area of a shape chosen from substantially polygonal, circular, oval, or irregular; and any combination of the above;
f. the power transmitted by said RF electrodes and said RF generating means to said skin is between 1 W and 700 W;
g. said cosmetic improvement is chosen from a group consisting of skin rejuvenation, reduction of the number of wrinkles, reduction of the depth of wrinkles, reduction of cellulite, skin tightening, circumferential reduction, and any combination of the above;
h. additionally comprising cooling means adapted to cool said skin;
i. said cooling means are chosen from a group consisting of a Peltier effect cooling device, irrigation with cool water, and means for blowing air across the skin;
j. said RF electrodes are further adapted to provide heat to said skin;
k. additionally comprising means for massaging said skin; and,
l. at least one of said RF electrodes comprises a hypodermic syringe for penetrating into subcutaneous tissue.

It is another object of the present invention to disclose the device, additionally comprising a deep tissue diathermy device.

It is another object of the present invention to disclose the device, wherein any of the following is held true:
a. said deep tissue diathermy device is chosen from a group consisting of any devices emitting RF radiation and any other means adapted for producing electrical current absorbed by subcutaneous tissue.
b. said deep tissue diathermy device further comprises:
   i. at least one electrical output device adapted to generate RF electromagnetic energy; and,
   ii. at least two electrodes electrically coupled to said electrical output device and placed on said skin region, wherein all said electrodes are adapted to simultaneously apply said RF energy to said skin.
c. said deep tissue diathermy device further comprises:
   i. at least one electrical output device adapted to generate electrical current; and,
   ii. at least two electrodes electrically coupled to said electrical output device and placed on said skin region, wherein all of said electrodes are adapted to simultaneously apply said electrical current to said skin.
d. said deep tissue diathermy device is chosen from a group consisting of acoustic (e.g., ultrasonic) diathermy devices, optical diathermy devices, electromagnetic induction diathermy devices, devices for producing sound waves, ultrasonic diathermy devices, and devices for direct application of heat.
e. said deep tissue diathermy device is an optical device adapted to emit light in wavelengths absorbed by subcutaneous tissue such that said subcutaneous tissue is heated;
f. said deep tissue diathermy device is a device for producing sound waves adapted to emit sound waves of a frequency absorbed by the subcutaneous tissue such that said subcutaneous tissue is heated;
g. said control means are adapted to monitor physical tissue parameters and to change at least one of (a) the amount of heat applied and (b) the form of said RF in response to the values of said physical tissue parameters;
h. said control means further comprise:
   i. processing means adapted to store in a communicable database predetermined parameters defining safe and unsafe treatment parameters, said parameters chosen from a group consisting of time of said treatment, the temperature of said skin, frequency, power, energy applied by said RF generating means, depth to which said device operates, magnetic field intensity, tissue impedance, specific absorption rate, superficial muscle contractions and any combination thereof;
   ii. sensing means adapted to sense electromagnetic radiation and heat radiation parameters chosen from a group consisting of time of said treatment, temperature of said tissue, frequency, power, intensity of ultrasound irradiation, energy applied by said RF generating means, depth to which said device operates, magnetic field intensity, tissue impedance, specific absorption rate, superficial muscle contractions and any combination thereof; and,
   iii. regulating means adapted to stop the operation of said device if said parameters are determined to be unsafe.

It is another object of the present invention to disclose the device, wherein said control means additionally comprise a feedback mechanism, adapted to change said RF signal according to predetermined medical needs, and comprising:
a. sensing means adapted to monitor electrotherapy parameters related to the level of skin rejuvenation and viability;
b. processing means, adapted to determine the degree of esthetic improvement in at least one tissue parameter related to the level of skin rejuvenation and vitality; and,
c. regulating means adapted to stop the operation of said device when said degree of esthetic improvement reaches a predetermined value.

It is another object of the present invention to disclose the device, wherein any of the following is held true:
a. said electrotherapy parameters are chosen from a group consisting of dermal tensile forces, tissue impedance, muscle contraction forces, skin elasticity, and any combination thereof.
b. said at least one tissue parameter is chosen from a group consisting of dermal tensile forces, tissue impedance, muscle contraction forces, skin elasticity, and any combination thereof.
c. wherein said sensing means are adapted to sense electrotherapy parameters are chosen from a group consisting of time of said treatment, temperature of said tissue, frequency, power, tissue impedance, superficial muscle contractions and any combination thereof.
d. said processing means are adapted to store in a communicable database predetermined parameters defining safe and unsafe treatment conditions; said predetermined parameters are chosen from a group consisting of time of said treatment, temperature of said tissue, frequency, power, tissue impedance, superficial muscle contractions, and any combination thereof.

It is another object of the present invention to disclose a method for providing cosmetic improvement to the skin, comprising:
a. providing a housing, said housing comprising: (i) N RF electrodes, N greater than or equal to 2, said RF electrodes configured to transmit RF energy to said skin so as to provide RF treatment to said skin; (ii) RF generating means for generating N/2 independent RF signals of predetermined waveforms, frequencies, and amplitudes, each of said N RF electrodes in independent communication with said RF generating means; (iii) at least one capsule, adapted to contain at least one medicament; said capsule within said housing; and (iv) M needles in fluid communication with said capsule; where M is greater than one;
b. generating at least one independent RF signal of predetermined waveform, frequency, and amplitude;
c. transmitting each of said independent RF signals to a pair of said electrodes;
d. placing said electrodes in physical contact with said skin; and,
e. transmitting energy carried by said RF signals to said skin, thereby providing said RF treatment; and
f. applying said medicament to said skin through at least one of said M needles
   wherein said medicament is applied to said skin either prior to or during RF treatment, such that said medicament is applied in conjunction with said RF treatment.

It is another object of the present invention to disclose the method, additionally comprising steps of providing control means for controlling the output of said RF generating means, said control means in communication with said RF generating means;

It is another object of the present invention to disclose the method, additionally comprising steps of delivering said medicament in conjunction with said RF in a manner selected from a group consisting of: medicament is delivered, then RF is applied, RF and medicament are applied simultaneously, RF is applied and then medicament, and any combination thereof.

It is another object of the present invention to disclose the method, additionally comprising steps of applying said RF energy to a subset of said N electrodes and changing said subset of said N electrodes with time, such that said electrodes provide fractional RF treatment of said skin.

It is another object of the present invention to disclose the method, additionally comprising steps of delivering said medicament through a subset of said M needles and changing said subset of said M needles with time, such that said needles provide fractional medicament treatment of said skin.

It is another object of the present invention to disclose the method, additionally comprising steps of changing said subset of said N electrodes and said subset of said M needles in conjunction with each other.

It is another object of the present invention to disclose the method, additionally comprising steps of selecting the shape of the distal end of said electrode from a group consisting of: pointed, cylindrical, rounded, conic, and truncated conic.

It is another object of the present invention to disclose the method, additionally comprising steps of providing said capsule frangible.

It is another object of the present invention to disclose the method, additionally comprising steps of squeezing said medicament from said housing onto said skin.

It is another object of the present invention to disclose the method, additionally comprising steps of flowing said medicament from said housing onto said skin.

It is another object of the present invention to disclose the method, additionally comprising steps of rupturing at least a portion of said capsule upon application of at least one of: at least one predetermined pressure on at least a portion the exterior of said housing, rotation of one part of said housing with respect to another part of said housing and any combination thereof.

It is another object of the present invention to disclose the method, additionally comprising steps of inducing fluid connection between the interior of said capsule and said skin via at least one said needle by said rupture.

It is another object of the present invention to disclose the method, wherein at least one of the following is true:
a. comprises steps of penetrating at least one at least one layer of said skin by at least one of said needles;
b. none of said needles penetrates any layer of said skin;
c. comprises steps of ablating a portion of at least one layer of said skin by at least one of said electrodes.

It is another object of the present invention to disclose the method, wherein any of the following is held true:
a. said steps of generating independent RF signals of predetermined waveforms, frequencies, and amplitudes comprise steps of generating independent RF signals with frequencies between about 1 Hz and about 100 MHz;
b. transmitting said RF signals either in a continuous mode or in pulses; when applying said RF in pulses, the length of said pulses is between about 0.01 μs and about 1 ms;
c. additionally comprising means adapted to apply Pulsed Electromagnetic Field Therapy (PEMF); said steps of transmitting said RF signals in pulses further comprising transmitting said pulses with pulse lengths of between about 0.1 and about 1000 ms;
d. additionally comprising steps of disposing said electrodes within an electrically insulating housing; said steps of disposing electrodes about the distal end of said housing in a geometry chosen from a group consisting of linear; zigzag; on the perimeter of a shape chosen from substantially polygonal, circular, oval, or irregular; within the area of a shape chosen from substantially polygonal, circular, oval, or irregular; and any combination of the above;
e. additionally comprising steps of transmitting to said skin a power of between 1 W and 700 W by said RF electrodes and said RF generating means;
f. additionally comprising steps of providing a cosmetic/esthetic improvement to the skin chosen from a group consisting of skin rejuvenation, reduction of the number of wrinkles, reduction of the depth of wrinkles, reduction of cellulite, skin tightening, circumferential reduction, and any combination of the above;
g. additionally comprising steps of cooling said skin; said steps of cooling said skin further comprising cooling said skin by the use of means chosen from a group consisting of a Peltier effect cooling device, irrigation with cool water, and means for blowing air across the skin;
h. additionally comprising steps of heating said skin by means of said RF electrodes;
i. additionally comprising steps of massaging said skin;
j. additionally comprising steps of incorporating a hypodermic syringe for penetrating into subcutaneous tissue into at least one of said electrodes.

It is another object of the present invention to disclose the method, additionally comprising steps of measuring the temperature of the surface of said skin.

It is another object of the present invention to disclose the method, wherein any of the following is held true:
a. said steps of measuring the temperature of the surface of said skin further comprise measuring the temperature of the surface of said skin by means of at least one sensor chosen from a group consisting of impedance meter adapted to measure impedance across at least one of pair of said RF electrodes; thermal sensor; thermometer; and any combination thereof;
b. said temperature measuring means either come into contact with said skin or are not in contact with said skin;
c. additionally comprising steps of regulating the amount of RF energy transmitted to said skin such that the temperature of said skin remains within a predetermined range; said steps of regulating the amount of RF energy transmitted to said skin further comprise regulating the amount of RF energy transmitted to said skin such that the temperature of said skin remains between ambient temperature and 42° C.; said steps of regulating said amount of RF energy transmitted to said skin further comprise regulating the amount of RF energy transmitted to said skin such that the temperature of said skin remains between 30° C. and 100° C.

It is another object of the present invention to disclose the method, additionally comprising steps of performing deep tissue diathermy; said steps of performing deep tissue diathermy further comprise performing deep tissue diathermy by a method chosen from a group consisting of: emitting RF radiation and producing electrical current absorbed by subcutaneous tissue.

It is another object of the present invention to disclose the method, wherein any of the following is held true:
a. said steps of performing deep tissue diathermy further comprise:
   i. generating RF electromagnetic energy by use of at least one electrical output device;
   ii. coupling at least two electrodes to said at least one electrical output device; and,
   iii. applying simultaneously said RF energy to said skin;
b. said steps of performing deep tissue diathermy further comprise:
   i. generating electrical current by means of at least one electrical output;
   ii. coupling electrically at least two electrodes electrically to said electrical output;
   iii. placing said electrodes on said skin; and,
   iv. applying simultaneously said electrical current to said skin;
c. said steps of performing deep tissue diathermy further comprise performing deep tissue diathermy by a means chosen from a group consisting of: ultrasonic diathermy devices, optical diathermy devices, electromagnetic induction diathermy devices, devices for producing sound waves, ultrasonic diathermy devices, and devices for direct application of heat;
d. said steps of performing deep tissue diathermy further comprise performing deep tissue diathermy by means of an optical device adapted to emit light in wavelengths absorbed by subcutaneous tissue such that said subcutaneous tissue is heated;
e. said steps of performing deep tissue diathermy further comprise performing deep tissue diathermy by means of a device for producing sound waves adapted to emit sound waves of a frequency absorbed by the subcutaneous tissue such that said subcutaneous tissue is heated;
f. additionally comprising steps of:
   i. monitoring physical tissue parameters; and,
   ii. changing at least one of (a) the amount of heat applied and (b) the form of said RF in response to the values of said physical tissue parameters;
g. additionally comprising steps of:
   i. storing in a communicable database predetermined parameters defining safe and unsafe treatment parameters, said parameters chosen from a group consisting of time of said treatment, the temperature of said skin, frequency, power, energy applied by said RF generating means, depth to which said device operates, magnetic field intensity, tissue impedance, specific absorption rate, superficial muscle contractions and any combination thereof;
   ii. sensing electromagnetic radiation and heat radiation parameters chosen from a group consisting of: time of said treatment, temperature of said tissue, frequency, power, intensity of ultrasound irradiation, energy applied by said RF generating means, depth to which said device operates, magnetic field intensity, tissue impedance, specific absorption rate, superficial muscle contractions and any combination thereof; and,
   iii. ceasing the continued performance of said method if said parameters are determined to be unsafe.

It is another object of the present invention to disclose the method, additionally comprising steps of:
a. monitoring electrotherapy parameters related to the level of skin rejuvenation and viability;
b. determining the degree of esthetic improvement in at least one tissue parameter related to the level of skin rejuvenation and vitality; and,
c. ceasing the continued performance of said method when said degree of esthetic improvement reaches a predetermined value.

It is another object of the present invention to disclose the method, wherein any of the following is held true:
a. said steps of monitoring electrotherapy parameters further comprise monitoring at least one parameter chosen from a group consisting of: dermal tensile forces, tissue impedance, muscle contraction forces, and skin elasticity.
b. said steps of determining the degree of esthetic improvement in at least one tissue parameter related to the level of skin rejuvenation and vitality further comprise determining the degree of esthetic improvement in at least one parameter chosen from a group consisting of: dermal tensile forces, tissue impedance, muscle contraction forces, and skin elasticity.
c. additionally comprising steps of monitoring electrotherapy parameters chosen from a group consisting of: time of said treatment, temperature of said tissue, frequency, power, tissue impedance, superficial muscle contractions and any combination thereof.
d. additionally comprising steps of storing in a communicable database predetermined parameters defining safe and unsafe treatment conditions, wherein said steps of storing in a communicable database said predetermined parameters defining safe and unsafe treatment conditions further comprise storing in a communicable database at least one parameter chosen from a group consisting of: time of said treatment, temperature of said tissue, frequency, power, tissue impedance, and superficial muscle contractions.

It is another object of the present invention to disclose a method for providing cosmetic improvement to the skin, comprising:
a. providing a housing, said housing comprising: (i) N RF electrodes, N greater than or equal to 2, said RF electrodes configured to transmit RF energy to said skin so as to provide RF treatment to said skin; (ii) RF generating means for generating N/2 independent RF signals of predetermined waveforms, frequencies, and amplitudes, each of said N RF electrodes in independent communication with said RF generating means; and (iii) at least one capsule, adapted to contain at least one medicament; said capsule within said housing; at least one of said N RF electrodes is in fluid communication with said capsule;
b. generating at least one independent RF signal of predetermined waveform, frequency, and amplitude;
c. transmitting each of said independent RF signals to a pair of said electrodes;
d. placing said electrodes in physical contact with said skin; and,
e. transmitting energy carried by said RF signals to said skin, thereby providing said RF treatment; and
f. applying said medicament to said skin through at least one of said N RF electrodes in fluid communication with said capsule
   wherein at least one of said N RF electrodes comprises a distal penetrating means adapted to penetrate said skin so as to deliver said medicament;
   further wherein said medicament is applied to said skin through at least one of said distal penetrating means either prior to or during said RF treatment, such that said medicament is applied in conjunction with said RF treatment.

It is another object of the present invention to disclose the method, additionally comprising steps of providing control means for controlling the output of said RF generating means, said control means in communication with said RF generating means;

It is another object of the present invention to disclose the method, additionally comprising steps of delivering said medicament in conjunction with said RF in a manner selected from a group consisting of: medicament is delivered, then RF is applied, RF and medicament are applied simultaneously, RF is applied and then medicament, and any combination thereof.

It is another object of the present invention to disclose the method, additionally comprising steps of applying said RF energy to a subset of said N electrodes and changing said subset of said N electrodes with time, such that said electrodes provide fractional RF treatment of said skin.

It is another object of the present invention to disclose the method, additionally comprising steps of delivering said medicament through a subset of said penetrating means and changing said subset of said penetrating means with time, such that said penetrating means provide fractional medicament treatment of said skin.

It is another object of the present invention to disclose the method, additionally comprising steps of changing said subset of said N electrodes and said subset of said penetrating means in conjunction with each other.

It is another object of the present invention to disclose the method, additionally comprising steps of selecting the shape of the distal end of said electrode from a group consisting of: pointed, cylindrical, rounded, conic, and truncated conic.

It is another object of the present invention to disclose the method, additionally comprising steps of providing said capsule frangible.

It is another object of the present invention to disclose the method, additionally comprising steps of squeezing said medicament from said housing onto said skin.

It is another object of the present invention to disclose the method, additionally comprising steps of flowing said medicament from said housing onto said skin.

It is another object of the present invention to disclose the method, additionally comprising steps of rupturing at least a portion of said capsule upon application of at least one of: at least one predetermined pressure on at least a portion the exterior of said housing, rotation of one part of said housing with respect to another part of said housing and any combination thereof.

It is another object of the present invention to disclose the method, additionally comprising steps of inducing fluid connection between the interior of said capsule and said skin via at least one said penetrating means by said rupture.

It is another object of the present invention to disclose the method, wherein at least one of the following is true:
a. comprises steps of penetrating at least one at least one layer of said skin by at least one of said penetrating means;
b. none of said penetrating means penetrates any layer of said skin;
c. comprises steps of ablating a portion of at least one layer of said skin by at least one of said electrodes.

It is another object of the present invention to disclose the method, wherein any of the following is held true:
a. said steps of generating independent RF signals of predetermined waveforms, frequencies, and amplitudes comprise steps of generating independent RF signals with frequencies between about 1 Hz and about 100 MHz;
b. transmitting said RF signals either in a continuous mode or in pulses; when applying said RF in pulses, the length of said pulses is between about 0.01 μs and about 1 ms;
c. additionally comprising means adapted to apply Pulsed Electromagnetic Field Therapy (PEMF); said steps of transmitting said RF signals in pulses further comprise transmitting said pulses with pulse lengths of between about 0.1 and about 1000 ms;
d. additionally comprising steps of disposing said electrodes within an electrically insulating housing; said steps of disposing electrodes about the distal end of said housing in a geometry chosen from a group consisting of linear; zigzag; on the perimeter of a shape chosen from substantially polygonal, circular, oval, or irregular; within the area of a shape chosen from substantially polygonal, circular, oval, or irregular; and any combination of the above;
e. additionally comprising steps of transmitting to said skin a power of between 1 W and 700 W by said RF electrodes and said RF generating means;
f. additionally comprising steps of providing a cosmetic/esthetic improvement to the skin chosen from a group consisting of skin rejuvenation, reduction of the number of wrinkles, reduction of the depth of wrinkles, reduction of cellulite, skin tightening, circumferential reduction, and any combination of the above;
g. additionally comprising steps of cooling said skin; said steps of cooling said skin further comprising cooling said skin by the use of means chosen from a group consisting of a Peltier effect cooling device, irrigation with cool water, and means for blowing air across the skin;
h. additionally comprising steps of heating said skin by means of said RF electrodes;
i. additionally comprising steps of massaging said skin;
j. additionally comprising steps of incorporating a hypodermic syringe for penetrating into subcutaneous tissue into at least one of said electrodes.

It is another object of the present invention to disclose the method, additionally comprising steps of measuring the temperature of the surface of said skin.

It is another object of the present invention to disclose the method, wherein any of the following is held true:
a. said steps of measuring the temperature of the surface of said skin further comprise measuring the temperature of the surface of said skin by means of at least one sensor chosen from a group consisting of impedance meter adapted to measure impedance across at least one of pair of said RF electrodes; thermal sensor; thermometer; and any combination thereof;
b. said temperature measuring means either come into contact with said skin or are not in contact with said skin;
c. additionally comprising steps of regulating the amount of RF energy transmitted to said skin such that the temperature of said skin remains within a predetermined range; said steps of regulating the amount of RF energy transmitted to said skin further comprise regulating the amount of RF energy transmitted to said skin such that the temperature of said skin remains between ambient temperature and 42° C.; said steps of regulating said amount of RF energy transmitted to said skin further comprise regulating the amount of RF energy transmitted to said skin such that the temperature of said skin remains between 30° C. and 100° C.

It is another object of the present invention to disclose the method, additionally comprising steps of performing deep tissue diathermy; said steps of performing deep tissue diathermy further comprise performing deep tissue diathermy by a method chosen from a group consisting of: emitting RF radiation and producing electrical current absorbed by subcutaneous tissue.

It is another object of the present invention to disclose the method, wherein any of the following is held true:
a. said steps of performing deep tissue diathermy further comprise:
   i. generating RF electromagnetic energy by use of at least one electrical output device;
   ii. coupling at least two electrodes to said at least one electrical output device; and,
   iii. applying simultaneously said RF energy to said skin;
b. said steps of performing deep tissue diathermy further comprise:
i. generating electrical current by means of at least one electrical output;
   ii. coupling electrically at least two electrodes electrically to said electrical output;
   iii. placing said electrodes on said skin; and,
   iv. applying simultaneously said electrical current to said skin;
c. said steps of performing deep tissue diathermy further comprise performing deep tissue diathermy by a means chosen from a group consisting of: ultrasonic diathermy devices, optical diathermy devices, electromagnetic induction diathermy devices, devices for producing sound waves, ultrasonic diathermy devices, and devices for direct application of heat;
d. said steps of performing deep tissue diathermy further comprise performing deep tissue diathermy by means of an optical device adapted to emit light in wavelengths absorbed by subcutaneous tissue such that said subcutaneous tissue is heated;
e. said steps of performing deep tissue diathermy further comprise performing deep tissue diathermy by means of a device for producing sound waves adapted to emit sound waves of a frequency absorbed by the subcutaneous tissue such that said subcutaneous tissue is heated;
f. additionally comprising steps of:
   i. monitoring physical tissue parameters; and,
   ii. changing at least one of (a) the amount of heat applied and (b) the form of said RF in response to the values of said physical tissue parameters;
g. additionally comprising steps of:
   i. storing in a communicable database predetermined parameters defining safe and unsafe treatment parameters, said parameters chosen from a group consisting of time of said treatment, the temperature of said skin, frequency, power, energy applied by said RF generating means, depth to which said device operates, magnetic field intensity, tissue impedance, specific absorption rate, superficial muscle contractions and any combination thereof;
   ii. sensing electromagnetic radiation and heat radiation parameters chosen from a group consisting of: time of said treatment, temperature of said tissue, frequency, power, intensity of ultrasound irradiation, energy applied by said RF generating means, depth to which said device operates, magnetic field intensity, tissue impedance, specific absorption rate, superficial muscle contractions and any combination thereof; and,
   iii. ceasing the continued performance of said method if said parameters are determined to be unsafe.

It is another object of the present invention to disclose the method, additionally comprising steps of:
a. monitoring electrotherapy parameters related to the level of skin rejuvenation and viability;
b. determining the degree of esthetic improvement in at least one tissue parameter related to the level of skin rejuvenation and vitality; and,
c. ceasing the continued performance of said method when said degree of esthetic improvement reaches a predetermined value.

It is another object of the present invention to disclose the method, wherein any of the following is held true:
a. said steps of monitoring electrotherapy parameters further comprise monitoring at least one parameter chosen from a group consisting of: dermal tensile forces, tissue impedance, muscle contraction forces, and skin elasticity.
b. said steps of determining the degree of esthetic improvement in at least one tissue parameter related to the level of skin rejuvenation and vitality further comprise determining the degree of esthetic improvement in at least one parameter chosen from a group consisting of: dermal tensile forces, tissue impedance, muscle contraction forces, and skin elasticity.
c. additionally comprising steps of monitoring electrotherapy parameters chosen from a group consisting of: time of said treatment, temperature of said tissue, frequency, power, tissue impedance, superficial muscle contractions and any combination thereof.
d. additionally comprising steps of storing in a communicable database predetermined parameters defining safe and unsafe treatment conditions, wherein said steps of storing in a communicable database said predetermined parameters defining safe and unsafe treatment conditions further comprise storing in a communicable database at least one parameter chosen from a group consisting of: time of said treatment, temperature of said tissue, frequency, power, tissue impedance, and superficial muscle contractions.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates an embodiment of the device of the present invention wherein electrodes comprise the medicament delivery means; and

FIG. 2 schematically illustrates an embodiment of the device of the present invention wherein electrodes apply the RF energy and separate hollow needles apply the medicament.

FIGS. 3A-E schematically illustrate the distal tip of an exemplary electrode.

FIGS. 4-5 schematically illustrate another embodiment of the present invention in which a combined IPL treatment is illustrated.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for treating a skin condition by applying RF energy and a medicament to the skin in conjunction with one another.

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the figures and described in the specification, but only as indicated in the accompanying claims, with the proper scope determined only by the broadest interpretation of said claims.

With respect to parameters that characterize the invention disclosed herein and the cosmetic treatment effected thereby, “unsafe” parameters are understood to be parameters that will cause tissue damage or excessive discomfort to the person undergoing treatment, e.g. overheating, transmitting energy to tissue layers below the skin, etc. According to one embodiment, the device of the present invention automatically prevents the parameters from reaching the unsafe zone and maintains the same within the safe zone.

The invention comprises a novel device for improving the cosmetic appearance of the skin, which is described in detail below. In preferred embodiments of the invention, the cosmetic improvement achieved includes at least one of skin rejuvenation; reduction in the number of wrinkles; reduction of the depth of wrinkles; reduction of cellulite; skin tightening; circumferential reduction.

Said cosmetic improvement is achieved by applying energy to the skin. The energy is selected from a group consisting of RF, Intense pulsed light (IPL), laser and any combination thereof.

The following disclosure is provided for RF energy, however the same applies both to laser or IPL treatment.

The invention provides an integrated mechanism for improving the cosmetic appearance of the skin. In the prior art the skin is treated with RF, then the RF applicator is removed and medicament is applied to the skin. Unlike the prior art, in the present invention, any of the following can be done, all without removing the device from the skin: medicament and RF can be applied simultaneously, or medicament can be applied, then RF, or RF then medicament, or RF and medicament can be applied alternately.

Furthermore, for the user, the device provides single step application of medicament and RF. In all embodiments, the user applies the device to the skin and operates the device, whether operation involves turning it on, squeezing it, or otherwise manipulating it. The user need not concern herself with the details of operation; the medicament will automatically be dispensed and the RF automatically applied as appropriate for the embodiment. For non-limiting example, in variants of embodiments where RF and medicament are applied simultaneously, each time the user squeezes the housing, medicament is dispensed onto the skin and RF is simultaneously applied to the skin.

The present invention provides a device for improving the cosmetic appearance of the skin, comprising a housing, said housing comprising:
a. N RF electrodes, where N is greater than two, said RF electrodes configured to transmit RF energy to said skin so as to provide RF treatment to said skin;
b. RF generating means for generating N/2 independent RF signals of predetermined waveforms, frequencies, and amplitudes; each of said N RF electrodes in independent communication with said RF generating means;
c. at least one capsule, adapted to contain at least one medicament; said capsule within said housing; and
d. M needles in fluid communication with said capsule; where M is greater than one;
wherein said medicament is applied to said skin through at least one of said M needles either prior to or during said RF treatment, such that said medicament is applied in conjunction with said RF treatment.

The present invention further provides a device for improving the cosmetic appearance of the skin, comprising a housing, said housing comprising:
a. N RF electrodes, where N is greater than two, said RF electrodes configured to transmit RF energy to said skin so as to provide RF treatment to said skin;
b. RF generating means for generating N/2 independent RF signals of predetermined waveforms, frequencies, and amplitudes; each of said N RF electrode in independent communication with said RF generating means; and
c. at least one capsule, adapted to contain at least one medicament; said capsule within said housing; at least one of said N RF electrodes is in fluid communication with said capsule;
   wherein at least one of said N RF electrodes comprises a distal penetrating means adapted to penetrate said skin so as to deliver said medicament; further wherein said medicament is applied to said skin through at least one of said distal penetrating means either prior to or during said RF treatment, such that said medicament is applied in conjunction with said RF treatment.

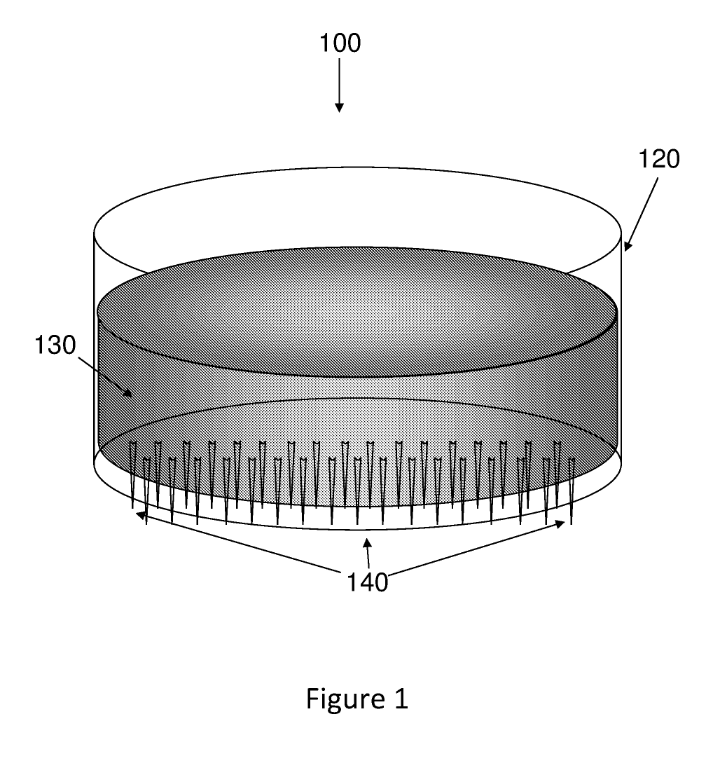

FIG. 1 schematically illustrates an embodiment (100) of the invention. The housing (120) contains a frangible medicament capsule (130). Depending from the base of the capsule are N electrodes (140) with associated electrical connections (not shown); each electrode (140) is independently connectable to a power source (not shown) and a control means (not shown). The proximal end of the electrodes (140) is within the housing (120) or on the interior of the housing (120) such that connection between the electrodes (140) and the housing (120) is fluid-tight. The distal end of the electrodes (140) is exterior to the housing (120).

At least a portion of the capsule (130) is frangible, such that exerting at least a predetermined pressure on the capsule (130) causes at least a portion of the capsule (130) to fracture, thereby allowing medicament to escape from the capsule and, as described hereinbelow, to flow onto the skin.

The predetermined pressure can be exerted by a method selected from a group consisting of: pressing the sides of the housing (120) toward each other; twisting a portion of the housing (120) relative to another portion, thereby compressing the capsule (130) between an upper anvil (which can be the top of the housing (120)) and the base of the housing (120); twisting a portion of the housing (120) relative to another portion, thereby pressing the capsule (130) against the tops of the electrodes (140), twisting a portion of the housing (120) relative to another portion, thereby pressing the capsule (130) against sharp objects in the base of the housing (120), or any combination thereof. Other methods of applying pressure to the capsule (120) will be obvious to one skilled in the art.

The electrodes (140) are adapted to deliver energy to the skin, preferably RF energy. The control means enables the energy to be applied to any subset of the electrodes, and to apply it in any desired pattern. For non-limiting example, energy can be applied to the electrodes (140) in random order, minimizing heating of the skin; or energy can be applied along a line of electrodes (140), sweeping along a wrinkle or scar while avoiding treating (or heating) unwrinkled skin. Other patterns of application will be obvious to one skilled in the art.

In some embodiments, the electrodes (140) are fixed in position.

In some embodiments, the electrodes (140) are extendible from the housing (120) such that, during storage and transport, the electrodes (140) are retracted within the housing (120) and are protected from damage. In some embodiments, the electrodes (140) can be retracted into the housing (120) after use, to ensure that the device can be disposed of without danger of someone being inadvertently scratched by the electrodes (140) during, for example, recycling processes.

In some embodiments, the electrodes (140) are hollow, open at their proximal end (within the housing (120)) and comprise a hole in the portion of the electrode (140) outside the housing (120), preferably near the distal end of the electrodes (140). These hollow electrodes (140) provide a fluid connection between at least a portion of the interior of the housing (120) and the exterior of the device, through which medicament can flow from the capsule (130) to the skin.

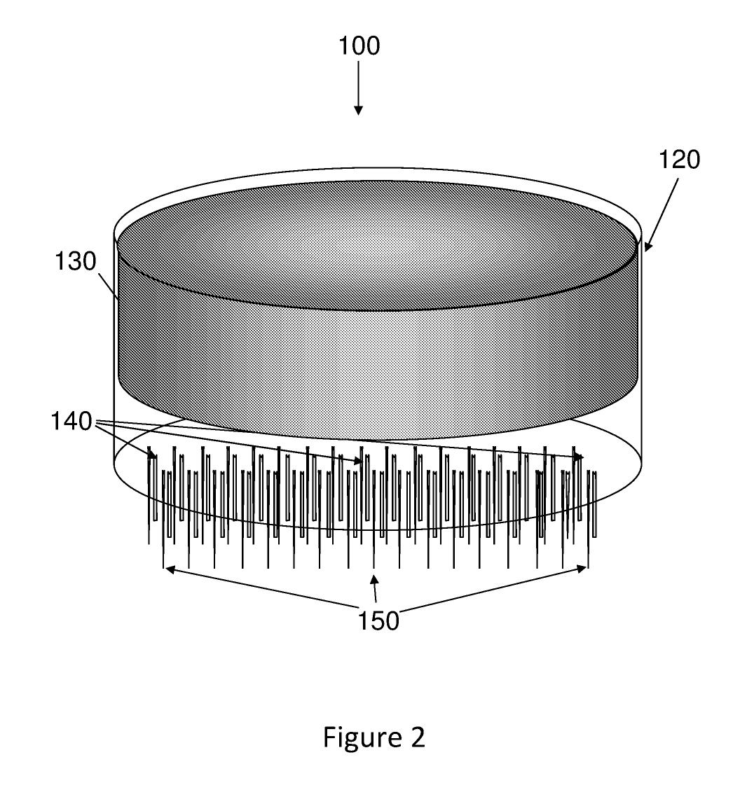

FIG. 2 schematically illustrates an embodiment (200) of the invention wherein the medicament is delivered to the skin via hollow needles (150). The housing (120) contains a frangible medicament capsule (130). Depending from the base of the capsule are N electrodes (140) with associated electrical connections (not shown); each electrode (140) is independently connectable to a power source (not shown) and a control means (not shown). The connection between the electrodes (140) and the housing (120) is fluid-tight. At least the distal end of the electrodes (140) is exterior to the housing (120).

In further reference to FIGS. 1 and 2, the capsule can be positioned anywhere within the housing.

In the exemplary embodiment of FIG. 1, the capsule is positioned in contact with or nearly in contact with the base of the housing. In some variants of embodiments where the capsule is in contact or nearly in contact with the base of the housing, at least one of the electrodes or needles can have sharp or serrated edges; in such embodiments, pressure on the housing pushes the capsule onto the sharp or serrated edges, thereby breaking the capsule and enabling flow of medicament into the hollow of the needle or electrode.

In the exemplary embodiment of FIG. 2, the capsule is positioned near the top of the housing. In embodiments of this type, after the capsule has fractured, the medicament will flow from the capsule into the base of the housing, providing an even layer of medicament, thereby assuring that the same amount of medicament is delivered through each of the hollow needles or electrodes.

In the exemplary embodiments shown, where it is present, the capsule fills the cross-section of the housing. In other embodiments, the capsule does not fill the entire cross-section; other part of the cross-section can contain, for non-limiting example, electrical connections, power sources, electronics, or piercing mechanisms.

In yet other embodiments, the capsules have more than one section, so that, for example, medicaments can be mixed at the time of use and kept separate before use.

Other positions of the capsule within the housing will be obvious to one skilled in the art.

Also depending from the base of the capsule (120) are P needles (150). The proximal end of the needles (150) is within the housing or on the interior of the housing such that connection between the needles (150) and the housing (120) is fluid-tight. The distal end of the needles (150) is exterior to the housing (120). The proximal end of the needles (150) is within the housing (120) or on the interior of the housing (120) such that connection between the needles (150) and the housing (120) is fluid-tight. The distal end of the needles (150) is exterior to the housing (120).

In preferred embodiments, the needles penetrate the skin. In some variants of these embodiments, the medicament is delivered to the skin below the surface of the skin. In other variants, the medicament is delivered to the skin at or above the level of the skin; in these embodiments, the medicament can flow into the skin via holes created by the penetrating needles.

In some embodiments, the electrodes induce holes in the skin, for non-limiting example, by ablation; in some variants of these embodiments, the medicament can flow into the skin via holes created by the electrodes.

In the embodiment of FIG. 2, rupture of the capsule (130) enables medicament to flow through the needles (150) to the skin.

In the embodiment of FIG. 2, fluid can flow through the needles (150) only; the electrodes' (140) function is to deliver energy to the skin.

In some variants of the embodiment of FIG. 2, fluid can flow through both the needles (150) and through the electrodes (140).

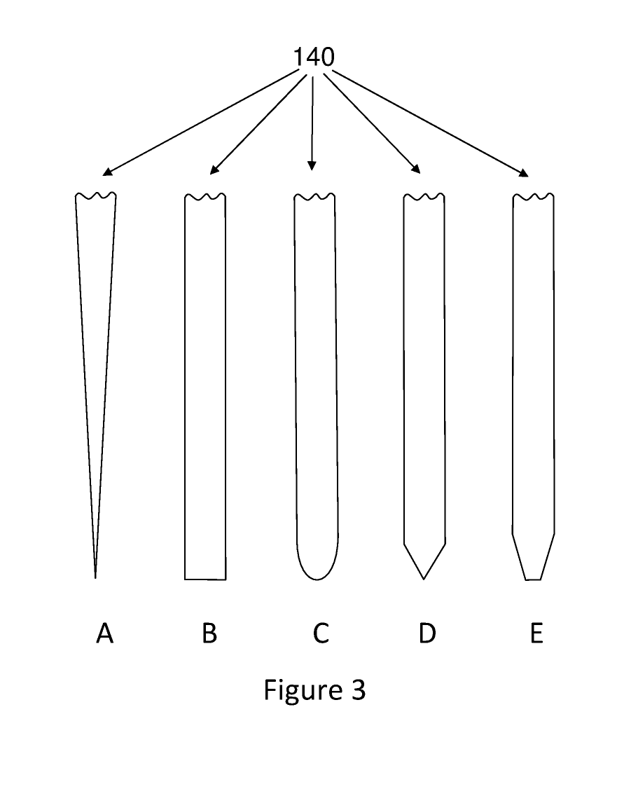

In reference to FIG. 3A-E, the distal tip of the electrode is shown. The distal tip of the electrode can be pointed (FIG. 3A, 3D), cylindrical (FIGS. 3B, 3E), or rounded (FIG. 3C). In embodiments such as those shown in FIGS. 3D and 3E, the electrode narrows only slowly, if it all, until near the tip, so that the tip is conic (FIG. 3D) or a truncated cone (FIG. 3E), also called the frustum of a cone. Near the tip, the narrowing is raped, so that an electrode with small tip area will still have sufficient thermal conductivity to prevent overheating.

If the electrode has circular cross-section, the distal tip in FIG. 3B will form a circular cylinder; if the electrode has polygonal cross-section, the cylinder will be polygonal.

In some embodiments, the medicament is squeezed from the housing; if there is no pressure on the housing, the medicament remains contained therein. In other embodiments, once the capsule has been ruptured, the medicament flows from the capsule through the needles or hollow electrodes to the skin, without requiring further pressure.

The medicament can be an analgesic or local anesthetic, to reduce discomfort caused by application of the energy, it can be a skin-treatment medicament such as, but not limited to, retinoids, Retinol, alpha hydroxy acids, kinetin, coenzyme Q10, copper peptides antioxidants, and topical vitamin C. Medicaments can also include, but are not limited to, a dye to absorb light in embodiments that use optical means for effecting deep tissue diathermy, a muscle relaxant, etc.

During treatment, the energy can be applied first, with medicament applied afterwards, the medicament can be applied first, with the energy applied afterward, the medicament and energy can be applied simultaneously, and any combination thereof.

The electrodes are configured to transmit RF radiation received from the RF source to the skin undergoing cosmetic treatment. The electrodes and electrical connections are enclosed within a housing (120) that is made of electrically non-conducting material such as, for example, plastic. In preferred embodiments, the electrodes are disposed about the distal end of the housing such that all of the electrodes can be placed simultaneously in physical contact with the skin undergoing cosmetic treatment. The geometrical arrangement of the electrodes can be any suitable arrangement; non-limiting examples include circular, linear, zigzag, on the perimeter and/or within the area of a substantially polygonal, circular, oval, or irregular shape, or any combination of the above.

Reference is now made to another embodiment of the present invention in which combined IPL treatment is illustrated.

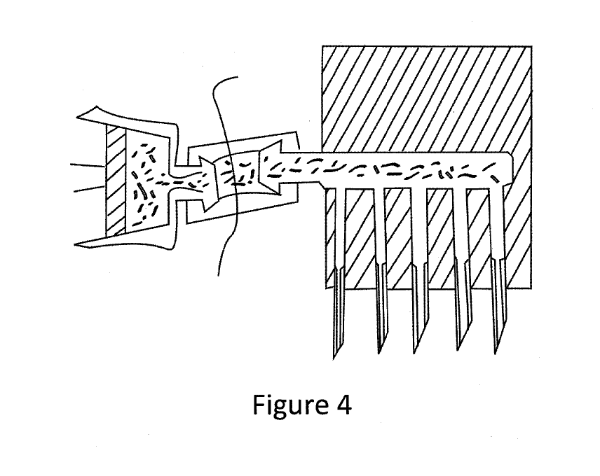

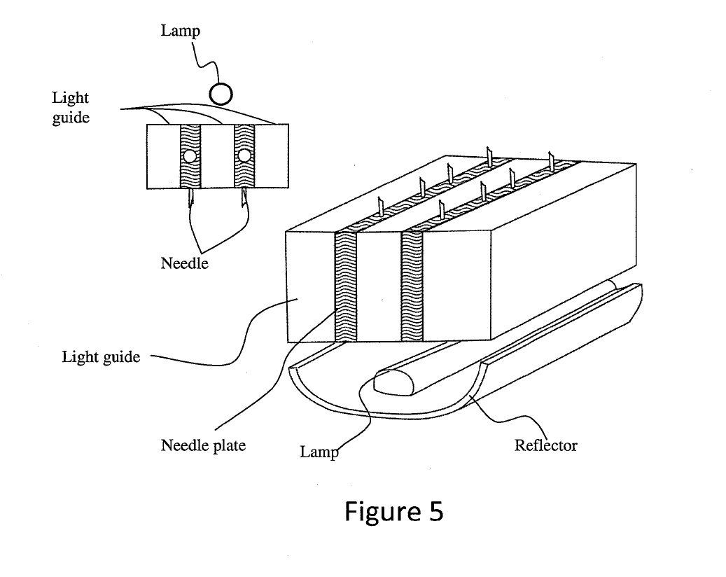

Reference is now made to FIGS. 4-5 which provides a preliminary sketch of the idea of combined IPL and infection treatment.

The IPL is a device utilizing the light from the flash lamp to treat the human tissue. The light usually delivered through the optical light guide which is a block of transparent material like sapphire or quartz.

In this embodiment, the light guides are made from few separate blocks and placing between the blocks the metal plates with micro needles.

The micro needles are connected inside of the metal plate in parallel. The metal plate connected to injection device by tubing.

According to this embodiment, the operation of the device is by positioning the light guide/metal plates sandwich on the tissue; pressing to penetrate into the skin; and, than to infect the some liquid with the pulse of light.

In order to ensure the user's safety, in preferred embodiments of the invention, the device is adapted to operate according to a medical electrical equipment standard chosen from a group consisting of IEC 60601-2-35, IEC 60601-2-33, IEC 60601-2-29, IEC 60601-2-9, IEC 60601-2-5, IEC 60601-2-3, IEC 60601-1-8, IEC 60601-1-6, IEC 60601-1-4, IEC 60601-1-3, IEC 60601-1-2, IEC 60601-1-1 and any combination thereof.

The RF generating means comprises means for producing N/2 independent RF signals of predetermined waveforms, frequencies, and amplitudes, (it should be noted that N is the number of electrodes). Means for production of RF signals with several independent output channels are well-known in the art. In preferred embodiments, the waveforms, frequencies, and amplitudes are chosen to be those that are most effective for skin treatment. In preferred embodiments, the frequencies of the RF signals are between about 1 Hz and about 100 MHz, and the power transmitted by said RF electrodes and said RF generating means to the skin undergoing cosmetic treatment is between 1 W and 700 W.

The RF output can be continuous wave or pulsed. In preferred embodiments in which the RF output is pulsed, the pulses have duration of between about 0.1 μsec and about 1 ms. However, it should be pointed that the RF can be applied in a continuous mode.

According to another embodiment of the present invention, there are provided means adapted to apply Pulsed Electromagnetic Field Therapy (PEMF). In preferred embodiments in which the RF output is pulsed, the pulses have duration of between about 0.1 ms and about 1000 ms.

At any given time, the number of pairs of electrodes which are active, which have current flowing through them, can be anywhere between 0 and N/2, where N is the total number of electrodes.

In some embodiments of the device, it also comprises means for measuring the temperature of the skin being treated.

In preferred embodiments, the temperature measuring is incorporated into housing 120 and disposed in such a manner that the temperature sensor makes contact with the skin while the device is in use.

In preferred embodiments of the invention that include a temperature sensor, the sensor is chosen from a group consisting of an impedance meter adapted to measure impedance across at least one of said pairs of RF electrodes; a thermal sensor; a thermometer; and any combination thereof.

In more preferred embodiments of the invention that include a temperature sensor, control means are programmed to regulate the amount of RF energy transmitted to the skin such that the temperature of the surface of the skin remains within a predetermined range. Feedback mechanisms for raising or lowering the output of a signal generator in response to and in correlation with an input from an external sensor and means for incorporating them into control mechanisms for signal generators are well-known in the art, and any such feedback mechanism appropriate for the control means can be used. In the most preferred embodiments that include a temperature sensor, the temperature range can be (a) from ambient temperature to 42° C. or (b) between 30° C. and 100° C.

It should be noted that according to one embodiment of the present invention, the temperature sensors are embedded within the device and according to another embodiment, the temperature sensors are position on the surface of the device.

According to some embodiments, the temperature sensors come in contact with the skin and according to other embodiments, the sensors are not in contact with the skin.

In other embodiments of the invention, the device incorporates cooling means adapted to cool the skin being treated. In preferred embodiments that incorporate such cooling means, the cooling means is selected from a group consisting of a Peltier effect cooling device, irrigation with cool water, and means for blowing air across the skin. The cooling means can be integrated into housing (120) or applied outside of the housing (120) either as a cooling unit attached to the housing (120) or as a separate unit.

In yet other embodiments of the invention, the RF electrodes are further adapted to provide heat to the skin being treated.

In some embodiments, the device according to the present invention additionally incorporates a device for performing deep tissue diathermy. In preferred embodiments of the invention that include a device for performing deep tissue diathermy, the device is selected from a group consisting of any devices emitting RF radiation and any other means adapted for producing electrical current absorbable by subcutaneous tissue.

In more preferred embodiments of the invention in which a deep tissue diathermy device is incorporated, the electrodes that provide the pulsed electromagnetic field (PEMF) therapy also provide RF electrical current for the diathermy treatment. Examples of how to incorporate deep tissue diathermy devices into a device for PEMF therapy are disclosed in detail in U. S. Pat. Appl. US2011/0130618; one of ordinary skill in the art will readily understand how to apply the information disclosed in that application to the device disclosed in the present invention.

In preferred embodiments of the invention in which it incorporates a deep tissue diathermy device, the deep tissue diathermy device further incorporates at least one electrical output device adapted to generate RF electromagnetic energy; and at least two electrodes electrically coupled to said electrical output device and placed on said skin region, wherein all said electrodes are adapted to simultaneously apply said RF energy to said skin.

In more preferred embodiments of the invention in which it incorporates a deep tissue diathermy device, the deep tissue diathermy device incorporates in addition at least one electrical output device adapted to generate electrical current; and at least two electrodes electrically coupled to said electrical output device and placed on said skin region, wherein any subset of said electrodes are adapted to simultaneously apply said electrical current to said skin, so that the deep tissue diathermy can be applied either to all of the tissue, to the tissue treated by RF, to the tissue treated by the medicament, to tissue different from the tissue treated by RF, to tissue different from the tissue treated by medicament, and any combination thereof, Tissue treated by diathermy can be treated in conjunction with RF treatment or medicament treatment, and the diathermy can be before, during or after the RF treatment, the medicament treatment and any combination thereof.

In the most preferred embodiments in which the device incorporates a deep tissue diathermy device, it is chosen from a group consisting of ultrasonic diathermy devices, optical diathermy devices, electromagnetic induction diathermy devices, devices for producing sound waves, acoustic diathermy devices, ultrasonic diathermy devices, and devices for direct application of heat.

In preferred embodiments in which the deep tissue diathermy device is an optical device, it is adapted to emit light in wavelengths absorbed by subcutaneous tissue such that said subcutaneous tissue is heated.

In other preferred embodiments in which the deep tissue diathermy device is a sonic or ultrasonic deep tissue diathermy device, it comprises a device for producing sound waves adapted to emit sound waves of a frequency absorbed by the subcutaneous tissue such that said subcutaneous tissue is heated. All of these means for performing deep tissue diathermy are well-known in the art, and any suitable means for adapting or accommodating them to the present invention can be used.

In yet other preferred embodiments of the invention, the control means further comprise processing means adapted to store in a communicable database predetermined parameters defining safe and unsafe treatment parameters, said parameters chosen from a group consisting of time t of said treatment, the temperature of said skin, frequency, power, energy applied by said RF generating means, depth to which said device operates, magnetic field intensity, tissue impedance, specific absorption rate, superficial muscle contractions and any combination thereof; sensing means adapted to sense electromagnetic radiation and heat radiation parameters chosen from a group consisting of time of said treatment, temperature of said tissue, frequency, power, intensity of ultrasound irradiation, energy applied by said RF generating means, depth to which said device operates, magnetic field intensity, tissue impedance, specific absorption rate, superficial muscle contractions and any combination thereof; and regulating means adapted to stop the operation of said device if said parameters are determined to be unsafe. Such measuring, processing, and regulating means are well-known in the art, and one skilled in the art will readily understand how to incorporate them into the present device.

In yet other preferred embodiments of the device, the control means further comprise a feedback mechanism adapted to change the RF signal according to predetermined medical needs and comprising: sensing means adapted to monitor electrotherapy parameters related to the level of skin rejuvenation and viability; processing means adapted to determine the degree of esthetic improvement in at least one tissue parameter related to the level of skin rejuvenation and vitality; and regulating means adapted to stop the operation of the device when the degree of esthetic improvement reaches a predetermined value. In more preferred embodiments, the electrotherapy parameters and/or the tissue parameters are chosen from a group consisting of dermal tensile forces, tissue impedance, muscle contraction forces, skin elasticity, and any combination thereof. In other preferred parameters, the sensing means are adapted to sense electrotherapy parameters chosen from a group consisting of duration of the treatment, temperature of the tissue, frequency, power, tissue impedance, superficial muscle contractions and any combination thereof. Means for measuring these parameters and for providing such a feedback mechanism and incorporating it into the device are well-known in the art.

In yet other preferred embodiments of the device in which the control means incorporate a feedback mechanism, the processing means are adapted to store in a communicable database predetermined parameters defining safe and unsafe treatment conditions. As above, “unsafe” treatment conditions include those in which tissue damage or discomfort to the person being treated is likely to result. In other preferred parameters, the predetermined parameters are chosen from a group consisting of duration of the treatment, temperature of the tissue, frequency, power, tissue impedance, superficial muscle contractions, and any combination thereof.

In yet other embodiments of the invention disclosed herein, it additionally comprises means for massaging said skin.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims, with the proper scope determined only by the broadest interpretation of the claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-218. (canceled)

219. A device for improving the cosmetic appearance of the skin, comprising a housing, said housing comprising:
a. N electrodes, where N is greater than two, said electrodes configured to transmit energy to said skin so as to provide treatment to said skin;
b. energy generating means for generating N/2 independent signals of predetermined waveforms, frequencies, and amplitudes; each of said N electrodes in independent communication with said energy generating means;
c. at least one capsule, adapted to contain at least one medicament; said capsule within said housing; and,
d. M needles in fluid communication with said capsule; where M is greater than one;
wherein said medicament is applied to said skin through at least one of said M needles either prior to or during said treatment, such that said medicament is applied in conjunction with said treatment;
further wherein said capsule is integrated within said housing.

220. The device of claim 219, wherein at least one of the following is being held true:
a. said energy is selected from a group consisting of RF, Intense pulsed light (IPL), laser and any combination thereof.
b. said device additionally comprises control means for controlling the output of said RF generating means, said control means in communication with said RF generating means;
c. said delivery of said medicament in conjunction with said RF is selected from a group consisting of: medicament is delivered, then RF is applied, RF and medicament are applied simultaneously, RF is applied and then medicament, and any combination thereof;
d. said electrodes provide fractional RF treatment of said skin, said RF energy being applied to said skin through a subset of said N electrodes, said subset of said N electrodes changing with time;
e. said needles provide fractional medicament treatment of said skin, the medicament being applied to the skin through a subset of said M needles, said subset of said M needles changing with time, and optionally said subset of said N electrodes and said subset of said M needles change in conjunction with each other;
f. the distal end of said electrode has a shape selected from a group consisting of: pointed, cylindrical, rounded, conic, and truncated conic;
g. said capsule is frangible;
h. said medicament is squeezed from said housing onto said skin;
i. said medicament flows from said housing onto said skin;
j. wherein at least a portion of said capsule is adapted such that at least a portion thereof ruptures upon application of at least one of: at least one predetermined pressure on at least a portion of the exterior of said housing, rotation of one part of said housing with respect to another part of said housing and any combination thereof, and optionally said capsule is adapted such said rupture induces fluid connection between the interior of said capsule and said skin via at least one said needle;
k. at least one of said needles penetrates at least one layer of said skin;
l. none of said needles penetrates any layer of said skin;
m. at least one of said electrodes ablates a portion of at least one layer of said skin;
n. each of said predetermined frequencies is between about 1 Hz and about 100 MHz; and,
o. RF signals are transmitted in either a continuous mode or in pulses, and optionally when said RF is applied in pulses, the length of said pulses is between about 0.1 μs and about 1 ms.

221. The device of claim 219, additionally comprising at least one of the following:
a. means adapted to apply Pulsed Electromagnetic Field Therapy (PEMF), and optionally, when said RF is applied in pulses, the length of said pulses is between about 0.1 ms and about 1000 ms; and,
b. temperature measuring means adapted to measure the temperature of the surface of said skin, wherein optionally, said temperature measuring means comprises at least one sensor chosen from a group consisting of: impedance meter adapted to measure impedance across at least one of pair of said RF electrodes; thermal sensor; thermometer; and any combination thereof, and wherein at least one of the following is being held true:
i. said temperature measuring means comes into contact with the skin;
ii. said temperature measuring means is not in contact with the skin; and,
iii. said control means are programmed to regulate the amount of RF energy transmitted to said skin such that the temperature of said skin remains within a predetermined range, where optionally said predetermined range is between 30° C. and 100° C., and most preferably between ambient temperature and 42° C.

222. The device of claim 219, wherein at least one of the following is being held true:
a. said housing is electrically insulating;
b. said electrodes are disposed about the distal end of said housing in a geometry chosen from a group consisting of: linear; zigzag; on the perimeter of a shape chosen from substantially polygonal, circular, oval, or irregular; within the area of a shape chosen from substantially polygonal, circular, oval, or irregular; and any combination of the above;
c. power transmitted by said RF electrodes and said RF generating means to said skin is between 1 W and 700 W;
d. said cosmetic improvement is chosen from a group consisting of skin rejuvenation, reduction of the number of wrinkles, reduction of the depth of wrinkles, reduction of cellulite, skin tightening, circumferential reduction, and any combination of the above;
e. said device additionally comprises cooling means adapted to cool said skin, and optionally, said cooling means are chosen from a group consisting of: a Peltier effect cooling device, irrigation with cool water, and a means for blowing air across the skin;
f. said RF electrodes are further adapted to provide heat to said skin;
g. said device additionally comprises means for massaging said skin; and,
h. at least one of said RF electrodes comprises a hypodermic syringe for penetrating into subcutaneous tissue.

223. The device according to claim 219, additionally comprising a deep tissue diathermy device, and wherein at least one of the following is being held true:
a. said deep tissue diathermy device is chosen from a group consisting of: a device emitting RF radiation and any other means adapted for producing electrical current absorbed by subcutaneous tissue;
b. said deep tissue diathermy device further comprises:
i. at least one electrical output device adapted to generate RF electromagnetic energy; and,
ii. at least two electrodes electrically coupled to said electrical output device and placed on said skin region, wherein all said electrodes are adapted to simultaneously apply said RF energy to said skin;
c. said deep tissue diathermy device further comprises:
i. at least one electrical output device adapted to generate electrical current; and,
ii. at least two electrodes electrically coupled to said electrical output device and placed on said skin region, wherein all of said electrodes are adapted to simultaneously apply said electrical current to said skin;
d. said deep tissue diathermy device is chosen from a group consisting of: acoustic (e.g., ultrasonic) diathermy devices, optical diathermy devices, electromagnetic induction diathermy devices, devices for producing sound waves, ultrasonic diathermy devices, and devices for direct application of heat;
e. said deep tissue diathermy device is an optical device adapted to emit light in wavelengths absorbed by subcutaneous tissue such that said subcutaneous tissue is heated;
f. said deep tissue diathermy device is a device for producing sound waves adapted to emit sound waves of a frequency absorbed by the subcutaneous tissue such that said subcutaneous tissue is heated; and,
g. said control means are adapted to monitor physical tissue parameters and to change at least one of (a) the amount of heat applied and (b) the form of said RF in response to the values of said physical tissue parameters.

224. The device of claim 219, wherein said control means further comprise:
a. processing means adapted to store in a communicable database predetermined parameters defining safe and unsafe treatment parameters, said parameters chosen from a group consisting of time of said treatment, the temperature of said skin, frequency, power, energy applied by said RF generating means, depth to which said device operates, magnetic field intensity, tissue impedance, specific absorption rate, superficial muscle contractions and any combination thereof;
b. sensing means adapted to sense electromagnetic radiation and heat radiation parameters chosen from a group consisting of time of said treatment, temperature of said tissue, frequency, power, intensity of ultrasound irradiation, energy applied by said RF generating means, depth to which said device operates, magnetic field intensity, tissue impedance, specific absorption rate, superficial muscle contractions and any combination thereof; and,
c. regulating means adapted to stop the operation of said device if said parameters are determined to be unsafe.

225. The device according to claim 219, wherein said control means additionally comprise a feedback mechanism, adapted to change said RF signal according to predetermined medical needs, and wherein at least one of the following is being held true:
a. said feedback mechanism comprises:
i. sensing means adapted to monitor electrotherapy parameters related to the level of skin rejuvenation and viability;
ii. processing means, adapted to determine the degree of said esthetic improvement in at least one tissue parameter related to the level of skin rejuvenation and vitality; and,
iii. regulating means adapted to stop the operation of said device when said degree of said esthetic improvement reaches a predetermined value;
b. wherein said electrotherapy parameters are chosen from a group consisting of: dermal tensile forces, tissue impedance, muscle contraction forces, skin elasticity, and any combination thereof;
c. said at least one tissue parameter is chosen from a group consisting of dermal tensile forces, tissue impedance, muscle contraction forces, skin elasticity, and any combination thereof;
d. said sensing means are adapted to sense electrotherapy parameters chosen from a group consisting of: time of said treatment, temperature of said tissue, frequency, power, tissue impedance, superficial muscle contractions and any combination thereof; and,
e. said processing means are adapted to store in a communicable database predetermined parameters defining safe and unsafe treatment conditions, and chosen from a group consisting of: time of said treatment, temperature of said tissue, frequency, power, tissue impedance, superficial muscle contractions, and any combination thereof.

226. A device for improving the cosmetic appearance of the skin, comprising a housing, said housing comprising:
a. N electrodes, where N is greater than two, said electrodes configured to transmit RF energy to said skin so as to provide treatment to said skin;
b. energy generating means for generating N/2 independent signals of predetermined waveforms, frequencies, and amplitudes; each of said N electrode in independent communication with said energy generating means; and,
c. at least one capsule, adapted to contain at least one medicament; said capsule within said housing; at least one of said N electrodes is in fluid communication with said capsule;
wherein at least one of said N electrodes comprises a distal penetrating means adapted to penetrate said skin so as to deliver said medicament;
further wherein said medicament is applied to said skin through at least one of said distal penetrating means either prior to or during said treatment, such that said medicament is applied in conjunction with said treatment;
further wherein said capsule is integrated within said housing.

227. The device of claim 226, wherein at least one of the following is being held true:
a. said energy is selected from a group consisting of RF, Intense pulsed light (IPL), laser and any combination thereof;
b. said device additionally comprises control means for controlling the output of said RF generating means, said control means in communication with said RF generating means;
c. said delivery of said medicament in conjunction with said RF is selected from a group consisting of: medicament is delivered, then RF is applied, RF and medicament are applied simultaneously, RF is applied and then medicament, and any combination thereof;
d. said electrodes provide fractional RF treatment of said skin, said RF energy being applied to the skin through a subset of said N electrodes, said subset of said N electrodes changing with time;
e. said penetrating means provide fractional medicament treatment of said skin, the medicament being applied to the skin through a subset of said penetrating means, said subset of said penetrating means changing with time, optionally said subset of said N electrodes and said subset of said penetrating means change in conjunction with each other;
f. the distal end of said electrode has a shape selected from a group consisting of: pointed, cylindrical, rounded, conic, and truncated conic;
g. said capsule is frangible;
h. said medicament is squeezed from said housing onto said skin; and,
i. said medicament flows from said housing onto said skin.

228. The device of claim 226, wherein at least one of the following is being held true:
a. at least a portion of said capsule is adapted such that at least a portion thereof ruptures upon application of at least one of: at least one predetermined pressure on at least a portion of the exterior of said housing, rotation of one part of said housing with respect to another part of said housing and any combination thereof;
b. said capsule is adapted such that said rupture induces fluid connection between the interior of said capsule and said skin via said at least one penetrating means;
c. at least one of said penetrating means penetrates at least one layer of said skin.
d. none of said penetrating means penetrates any layer of said skin;
e. at least one of said electrodes ablates a portion of at least one layer of said skin;
f. each of said predetermined frequencies is between about 1 Hz and about 100 MHz;
g. RF signals are transmitted in either a continuous mode or in pulses and optionally, when said RF is applied in pulses, the length of said pulses is between about 0.1 μs and about 1 ms; and,
h. said device additionally comprises means adapted to apply Pulsed Electromagnetic Field Therapy (PEMF) and optionally, when said RF is applied in pulses, the length of said pulses is between about 0.1 ms and about 1000 ms.

229. The device of claim 226, wherein said device additionally comprising temperature measuring means adapted to measure the temperature of the surface of said skin, and wherein at least one of the following is being held true:
a. wherein said temperature measuring means comprise at least one sensor chosen from a group consisting of: impedance meter adapted to measure impedance across at least one of pair of said RF electrodes; thermal sensor; thermometer; and any combination thereof;
b. said temperature measuring means comes into contact with the skin;
c. said temperature measuring means is not in contact with the skin; and,
d. wherein said control means are programmed to regulate the amount of RF energy transmitted to said skin such that the temperature of said skin remains within a predetermined range, optionally said predetermined range is between 30° C. and 100° C., and most preferably between ambient temperature and 42° C.

230. The device of claim 226, wherein at least one of the following is being held true:
a. said housing is electrically insulating;
b. said electrodes are disposed about the distal end of said housing in a geometry chosen from a group consisting of linear; zigzag; on the perimeter of a shape chosen from substantially polygonal, circular, oval, or irregular; within the area of a shape chosen from substantially polygonal, circular, oval, or irregular; and any combination of the above;
c. the power transmitted by said RF electrodes and said RF generating means to said skin is between 1 W and 700 W;
d. said cosmetic improvement is chosen from a group consisting of skin rejuvenation, reduction of the number of wrinkles, reduction of the depth of wrinkles, reduction of cellulite, skin tightening, circumferential reduction, and any combination of the above;
e. said device additionally comprises cooling means adapted to cool said skin and said cooling means are chosen from a group consisting of: a Peltier effect cooling device, irrigation with cool water, and means for blowing air across the skin;
f. said RF electrodes are further adapted to provide heat to said skin;
g. said device additionally comprises means for massaging said skin; and,
h. at least one of said RF electrodes comprises a hypodermic syringe for penetrating into subcutaneous tissue.

231. The device according to claim 226, additionally comprising a deep tissue diathermy device, and wherein at least one of the following is being held true:
a. said deep tissue diathermy device is chosen from a group consisting of a device emitting RF radiation and any other means adapted for producing electrical current absorbed by subcutaneous tissue;
b. said deep tissue diathermy device further comprises:
i. at least one electrical output device adapted to generate RF electromagnetic energy; and,
ii. at least two electrodes electrically coupled to said electrical output device and placed on said skin region, wherein all said electrodes are adapted to simultaneously apply said RF energy to said skin;
c. said deep tissue diathermy device further comprises:
i. at least one electrical output device adapted to generate electrical current; and,
ii. at least two electrodes electrically coupled to said electrical output device and placed on said skin region, wherein all of said electrodes are adapted to simultaneously apply said electrical current to said skin;
d. said deep tissue diathermy device is chosen from a group consisting of acoustic (e.g., ultrasonic) diathermy devices, optical diathermy devices, electromagnetic induction diathermy devices, devices for producing sound waves, ultrasonic diathermy devices, and devices for direct application of heat.
e. said deep tissue diathermy device is an optical device adapted to emit light in wavelengths absorbed by subcutaneous tissue such that said subcutaneous tissue is heated;
f. said deep tissue diathermy device is a device for producing sound waves adapted to emit sound waves of a frequency absorbed by the subcutaneous tissue such that said subcutaneous tissue is heated; and,
g. said control means are adapted to monitor physical tissue parameters and to change at least one of (a) the amount of heat applied and (b) the form of said RF in response to the values of said physical tissue parameters.

232. The device of claim 225, wherein said control means further comprise:
a. processing means adapted to store in a communicable database predetermined parameters defining safe and unsafe treatment parameters, said parameters chosen from a group consisting of time of said treatment, the temperature of said skin, frequency, power, energy applied by said RF generating means, depth to which said device operates, magnetic field intensity, tissue impedance, specific absorption rate, superficial muscle contractions and any combination thereof;
b. sensing means adapted to sense electromagnetic radiation and heat radiation parameters chosen from a group consisting of time of said treatment, temperature of said tissue, frequency, power, intensity of ultrasound irradiation, energy applied by said RF generating means, depth to which said device operates, magnetic field intensity, tissue impedance, specific absorption rate, superficial muscle contractions and any combination thereof; and,
c. regulating means adapted to stop the operation of said device if said parameters are determined to be unsafe.

233. The device of claim 225, wherein said control means additionally comprise a feedback mechanism, adapted to change said RF signal according to predetermined medical needs, and wherein said feedback mechanism comprises:
a. sensing means adapted to monitor electrotherapy parameters related to the level of skin rejuvenation and viability; and optionally said electrotherapy parameters are chosen from a group consisting of dermal tensile forces, tissue impedance, muscle contraction forces, skin elasticity, and any combination thereof
b. processing means, adapted to determine the degree of said esthetic improvement in at least one tissue parameter related to the level of skin rejuvenation and vitality; and,
c. regulating means adapted to stop the operation of said device when said degree of said esthetic improvement reaches a predetermined value.
d. said at least one tissue parameter is chosen from a group consisting of dermal tensile forces, tissue impedance, muscle contraction forces, skin elasticity, and any combination thereof;
e. said sensing means are adapted to sense electrotherapy parameters;
f. said sensing means adapted to sense electrotherapy parameters are chosen from a group consisting of: time of said treatment, temperature of said tissue, frequency, power, tissue impedance, superficial muscle contractions and any combination thereof;
g. said processing means are adapted to store in a communicable database predetermined parameters defining safe and unsafe treatment conditions; and,
h. said predetermined parameters are chosen from a group consisting of: time of said treatment, temperature of said tissue, frequency, power, tissue impedance, superficial muscle contractions, and any combination thereof.

234. A method for providing cosmetic improvement to the skin, comprising:
i. providing a housing, said housing comprising: (i) N electrodes, N greater than or equal to 2, said electrodes configured to transmit energy to said skin so as to provide treatment to said skin; (ii) energy generating means for generating N/2 independent signals of predetermined waveforms, frequencies, and amplitudes, each of said N electrodes in independent communication with said generating means; (iii) at least one capsule, adapted to contain at least one medicament; said capsule within said housing; and (iv) M needles in fluid communication with said capsule; where M is greater than one;
ii. generating at least one independent signal of predetermined waveform, frequency, and amplitude;
iii. transmitting each of said independent signals to a pair of said electrodes;
iv. placing said electrodes in physical contact with said skin;
v. transmitting energy carried by said signals to said skin, thereby providing said treatment; and,
vi. applying said medicament to said skin through at least one of said M needles wherein said medicament is applied to said skin either prior to or during RF treatment, such that said medicament is applied in conjunction with said treatment;
further wherein said capsule is integrated within said housing.

235. The method of claim 229, additionally comprising at least one of the following steps:
a. Selecting said energy to be from a group consisting of RF, Intense pulsed light (IPL), laser and any combination thereof.
b. providing control means for controlling the output of said RF generating means, said control means in communication with said RF generating means;
c. delivering said medicament in conjunction with said RF in a manner selected from a group consisting of: medicament is delivered, then RF is applied, RF and medicament are applied simultaneously, RF is applied and then medicament, and any combination thereof;
d. applying said RF energy to a subset of said N electrodes and changing said subset of said N electrodes with time, such that said electrodes provide fractional RF treatment of said skin;
e. delivering said medicament through a subset of said M needles and changing said subset of said M needles with time, such that said needles provide fractional medicament treatment of said skin, and optionally changing said subset of said N electrodes and said subset of said M needles in conjunction with each other;

236. The method of claim 229, additionally comprising at least one of the following steps:
a. selecting an electrically insulating housing;
b. disposing said electrodes are about the distal end of said housing in a geometry chosen from a group consisting of: linear; zigzag; on the perimeter of a shape chosen from substantially polygonal, circular, oval, or irregular; within the area of a shape chosen from substantially polygonal, circular, oval, or irregular; and any combination of the above;
c. transmitting to said skin a power of between 1 W and 700 W by said RF electrodes and said RF generating means;
d. selecting said cosmetic/esthetic improvement to said skin from a group consisting of:
skin rejuvenation, reduction of the number of wrinkles, reduction of the depth of wrinkles, reduction of cellulite, skin tightening, circumferential reduction, and any combination of the above;
e. cooling said skin and optionally, said steps of cooling said skin are selected from a group consisting of: a Peltier effect cooling device, irrigation with cool water, and means for blowing air across the skin;
f. heating said skin by means of said RF electrodes;
g. massaging said skin;
h. incorporating a hypodermic syringe for penetrating into subcutaneous tissue into at least one of said electrodes; and,
i. disposing said electrodes about the distal end of said housing in a geometry chosen from a group consisting of linear; zigzag; on the perimeter of a shape chosen from substantially polygonal, circular, oval, or irregular; within the area of a shape chosen from substantially polygonal, circular, oval, or irregular; and any combination of the above.

237. A method for providing cosmetic improvement to the skin, comprising:
i. providing a housing, said housing comprising: (i) N electrodes, N greater than or equal to 2, said electrodes configured to transmit energy to said skin so as to provide RF treatment to said skin; (ii) energy generating means for generating N/2 independent signals of predetermined waveforms, frequencies, and amplitudes, each of said N electrodes in independent communication with said generating means; and (iii) at least one capsule, adapted to contain at least one medicament; said capsule within said housing; at least one of said N electrodes is in fluid communication with said capsule;
ii. generating at least one independent signal of predetermined waveform, frequency, and amplitude;
iii. transmitting each of said independent signals to a pair of said electrodes;
iv. placing said electrodes in physical contact with said skin;
v. transmitting energy carried by said signals to said skin, thereby providing said treatment; and,
vi. applying said medicament to said skin through at least one of said N RF electrodes in fluid communication with said capsule
wherein at least one of said N electrodes comprises a distal penetrating means adapted to penetrate said skin so as to deliver said medicament;
further wherein said medicament is applied to said skin through at least one of said distal penetrating means either prior to or during said treatment, such that said medicament is applied in conjunction with said treatment;
further wherein said capsule is integrated within said housing.

238. The method of claim 234, additionally comprising at least one of the following steps:
a. Selecting said energy to be from a group consisting of RF, Intense pulsed light (IPL), laser and any combination thereof.
b. providing control means for controlling the output of said RF generating means, said control means in communication with said RF generating means;
c. delivering said medicament in conjunction with said RF in a manner selected from a group consisting of: medicament is delivered, then RF is applied, RF and medicament are applied simultaneously, RF is applied and then medicament, and any combination thereof;
d. applying said RF energy to a subset of said N electrodes and changing said subset of said N electrodes with time, such that said electrodes provide fractional RF treatment of said skin;
e. delivering said medicament through a subset of said penetrating means and changing said subset of said penetrating means with time, such that said penetrating means provide fractional medicament treatment of said skin;
f. changing said subset of said N electrodes and said subset of said penetrating means in conjunction with each other;
